(19) United States
(12) Patent Application Publication
(10) Pub. No.: US 2026/0108365 A1
(43) Pub. Date: Apr. 23, 2026

(54) FORCE-LIMITING TRIALS

(71) Applicant: NEXUS SPINE, LLC, Salt Lake City, UT (US)

(72) Inventors: Peter A. Halverson, Salt Lake City, UT (US); David T. Hawkes, Salt Lake City, UT (US)

(21) Appl. No.: 19/415,269

(22) Filed: Dec. 10, 2025

Related U.S. Application Data
(63) Continuation-in-part of application No. 18/238,216, filed on Aug. 25, 2023, now Pat. No. 12,558,084.
(60) Provisional application No. 63/401,104, filed on Aug. 25, 2022.

(51) Int. Cl. A61F 2/46

(52) U.S. Cl. CPC A61F 2/4611; A61F 2002/4622; A61F 2002/4658; A61F 2002/4666

(57) ABSTRACT

Systems and methods for applying a spinal manipulation force, and measuring the spinal manipulation force or a resultant displacement, are provided. Some implementations of the systems and methods include a manipulation instrument having one or more manipulators or gauges. In some cases, the manipulator is configured to apply the spinal manipulation force to a spine of a patient. In some cases, the gauge is configured to measure one or more of the spinal manipulation force and the resultant displacement. Other implementations are discussed herein.

### RELATED APPLICATIONS AND PRIORITY CLAIMS

This is a continuation-in-part of, and claims priority to, U.S. Patent Application No. 18/238,216 (Attorney Docket No. 23845.155), filed August 25, 2023, and entitled SYSTEMS AND METHODS FOR MEASURING AND APPLYING A SPINAL COMPRESSION FORCE, which in turn claims priority to U.S. Provisional Patent Application Ser. No. 63/401,104 (Attorney Docket No. 23845.151), filed August 25, 2022, and entitled “METHODS AND INSTRUMENTS FOR APPLYING COMPRESSION TO THE SPINE”. The disclosures of each of the foregoing is incorporated herein by reference in their entireties.

### FIELD

The present disclosure relates to spinal manipulation, and more particularly to systems and methods for measuring and applying a spinal manipulation force.

### BACKGROUND AND RELATED ART

Treatment of spinal conditions often requires spinal surgery, such as spinal fusion surgery and total disc replacement surgery. During some spinal procedures, the spine may be distracted or compressed to allow implantation of interbody devices or artificial intervertebral discs, or to surgically alter the soft or hard tissues surrounding the spine. In some such instances, it is often desirable to apply manipulation or extension to the spine. For example, proper application of manipulation or extension may help facilitate bone growth, correct deformities (e.g., loss of segmental height, deformities caused by trauma, deformities caused by scoliosis, and other deformities), and lead to a proper sagittal balance through correction of lordosis (inward curve of the lumbar spine) or kyphosis (exaggerated forward rounding of the upper back).

In many cases, one or more spinal implants are affixed to or inserted between a patient’s spinal vertebrae to apply the desired degree of manipulation. However, determining the proper size of implant to use (or the optimal distance to separate spinal vertebrae from one another) can be difficult for several reasons. For example, the proper space between vertebrae may differ based on many factors, such as: the location of the vertebrae within the spine (e.g., the spinal segments in which the vertebrae are located); the stiffness of the vertebrae; the stiffness of the surrounding tissues; the amount of muscle, fat, and other tissue surrounding the vertebrae (especially where tissue has been removed as part of the operation in question); the size of the spinal vertebrae; and the individual anatomy of the patient in question. Accordingly, vertebrae separation (e.g., the implant size) is often determined subjectively. In some cases, a physician applies a subjective amount of force to spinal vertebrae to “get a feel for” the proper size of implant to use. In some cases, a physician hammers (or otherwise inserts) “trials” representing various sized implants between the vertebrae, then subjectively determines which size of implant to use based on the physician’s opinion about which trial fits best. The fracture strength of the bone is often less than the rupture strength of the surrounding elastic ligaments and cartilage. Thus, subjectively wedging a trial into the disc space can, in some cases, commonly fracture endplates and cause pain.

During the process of determining the correct spinal implant configuration, many forces are often applied to the spine of the patient. For example, a physician may squeeze (or otherwise actuate) a distraction tool to apply a force. Vibration and sharp forces may also be applied when a physician is hammering trials into the patient’s spine. These forces can cause damage to the spine, surrounding tissue, or other parts of the patient’s body, thereby causing pain, disfunction, or other problems (which may be significant and long lasting, or even permanent).

Moreover, using an improper implant configuration (e.g., selecting a size of implant that is too large or too small can place too much pressure or provide too little stability between vertebrae) can lead to bone fracture, bone resorption, lack of bone growth, and other severe problems. Thus, spinal operations can, in some cases, cause more problems than they solve.

Thus, while there are known techniques for treating spinal conditions, such techniques are not necessarily without their shortcomings, particularly (as described above) with relation to spinal fusion surgery, total disc replacement, spinal manipulation, and selection of proper spinal implant configurations. Accordingly, it would be an improvement in the art to augment or even replace current techniques with other techniques.

### BRIEF SUMMARY

Systems and methods for measuring and applying one or more spinal manipulation forces are provided. Some implementations of the systems and methods include a manipulation instrument having one or more manipulators or gauges. In some cases, the manipulator is configured to apply the spinal manipulation force to a spine of a patient. In some cases, the gauge is configured to measure the spinal manipulation force.

In some implementations of the manipulation instrument, the manipulator includes one or more arms, such as a first arm and a second arm (and any number of additional arms). In some cases, the first arm includes a first tip and a first handle. In some cases, the second arm includes a second tip and a second handle. Accordingly, some implementations of the manipulation instrument generally resemble a pair of calipers, scissors, pliers, reverse pliers, forceps, hemostats, rongeurs, shears, clippers, grippers, or any other suitable two-armed tool. In some instances, applying a force to at least one of the first handle and the second handle causes at least one of the first tip and the second tip to move apart to apply the spinal manipulation force to the spine.

In some implementations, at least one of the first tip and the second tip is configured to selectively couple to one or more spinal implants. For example, in some implementations, the tip (e.g., either or both) is shaped and sized to correspond to an anchor (e.g., a screw, nail, bolt, rivet, pin, plate, implant, shaft, or any other component configured to be attached to a patient’s spine). In some implementations, one or more of the tips are configured to contact, couple directly to, or otherwise interface with the spine itself (e.g., by having a surface sized and shaped appropriately to contact a surface of a vertebra).

Some implementations of the described instrument having multiple arms include one or more pivots, pivot joints, or other joints. In some cases, the first arm and the second arm are movably coupled together to form the pivot or movable joint. Some implementations include a pivot coupler (e.g., a screw, bolt, nail, rod, thread, hinge, articulation, axis, joint, bearing, ball-and socket-joint, or any other suitable type of fastener or mechanism that could be used to couple multiple objects together to form a fulcrum, pivot, hinge, axle, spindle, or other static or dynamic connection point). In some implementations, the first arm is coupled to the second arm (e.g., via the pivot) such that when the first arm is biased toward the second arm (e.g., when a user squeezes the handles toward each other), the first tip is biased toward the second tip (e.g., the tips come together, as with traditional scissors). Some implementations of the first arm are coupled to the second arm (e.g., via the pivot) such that when the first arm is biased toward the second arm, the first tip is biased away from the second tip (e.g., the tips diverge, as with reverse pliers).

While the gauge can include any suitable component for measuring or conveying the manipulation force applied by the manipulation instrument, the distance between the vertebrae as a result of such force, or any other measurement, some implementations of the gauge include one or more indicators. In some cases, the indicator is configured to convey a measurement of the spinal manipulation force to a user. While the indicator may include any component suitable for conveying such a measurement, some implementations of the indicator include one or more needles, rods, shafts, arrows, sliders, dials, lasers, lights, displays, or other components configured to dynamically shift (e.g., change position, orientation, size, color, or any other perceivable characteristics) to display, emit, or otherwise convey one or more measurement values.

In some implementations, the manipulator is configured to apply a set, known amount of force (such as an amount of force that is considered to be safe to apply to the spine without damaging the vertebrae or other spinal structures). In some cases, the gauge is configured to measure the distance between the vertebrae (or the displacement of the vertebrae) as a result of the application of the set spinal manipulation force.

Although the gauge can be positioned anywhere with respect to the manipulator, some implementations of the gauge are positioned between or adjacent to the first arm and the second arm. Some iterations of the gauge are configured to move along a proximal-distal axis. Indeed, some implementations are configured to be allowed to move the gauge a certain distance distally, with such distance growing larger as a distance between the first tip and the second tip increases. In some implementations, a distal end of the gauge thus moves between the vertebrae (in some cases, without touching the vertebrae) or between a portion of the first tip and the second tip, thereby causing a proximal end of the gauge to move distally as well. In some cases, the proximal end (or any other suitable portion) includes the indicator (e.g., with a scale or indicia) to show how far the gauge has moved, thereby conveying a measurement of the distance between vertebrae to a user.

According to some implementations, the manipulator includes (instead of or in addition to any of the features mentioned above or elsewhere herein) one or more housings. While the housing can include any component configured to form a particular shape or to house one or more other components, some embodiments of the housing are sized, shaped, and otherwise configured to be inserted into a spine. In some cases, the housing is configured to be inserted into a space between two vertebrae of the spine of the patient. Indeed, some iterations of the housing are configured to generally resemble a spinal implant trial. Some implementations include a trial arm (or guide), which, in some cases, is coupled or couplable to the housing.

In some implementations, the gauge includes one or more manipulation discs, which in some cases are optionally disposed within the housing. Although some implementations have only a single manipulation disc, some implementations include multiple manipulation discs. In some cases, the manipulation discs form a stack of discs.

In some implementations, one or more manipulation discs include one or more curved or angled surfaces. In some cases, one or more of the curved or angled surfaces are configured to flatten or become flatter when compressed. To illustrate, some implementations of the manipulation discs generally resemble a plate (or a disc, ring, sheet, leaf, leaf spring, polygon, scalloped polygon, implant shaped object, kidney shaped object, toroidal object, asymmetrical object, symmetrical object, or any other component that can be used with the implant) that is bent, bowed, angled, waved, or flexed, or that curves, ripples, undulates, cups, or otherwise forms a shape that is adapted to change configuration when compressed. Thus, when the manipulation discs are compressed, the change in height of the manipulation discs (including a change in any particular manipulation disc on its own, or a change in a stack or collection of manipulation discs in the conglomerate) can be measured to determine a force applied to the gauge. In some cases, the manipulation discs are pre-calibrated to determine a precise force that correlates with a particular amount of change or deformation.

In some implementations having multiple manipulation discs, a first manipulation disc and a second manipulation disc are situated (e.g., within the housing) so that the curved or angled surface of the first manipulation disc faces a first direction and the curved or angled surface of the second manipulation disc faces a second direction. In some embodiments, the first disc faces with the concave curved or angled surface pointed in a downward or posterior direction (e.g., away from a lid of the housing) while the second disc faces with the concave curved surface pointed in an upward or anterior direction (e.g., toward a lid of the housing). In this manner, one or more gaps between the two discs (in a resting configuration) can be maximized (or otherwise configured) so that a change in height of the stack, when compressed, is more pronounced. In some instances, multiple manipulation discs are directly adjacent to each other, or even in contact with one another, whereas in some instances, multiple manipulation discs are present within the housing (or without) but are separated from each other by one or more barriers or another component.

In some implementations, the discs profile or shape can be a shape other than being round or circular. For example, some implementations of discs are shaped to match or otherwise correspond with the shape of an implant, or the shape of the vertebrae, which are not necessarily round or circular. More specifically, some implementations of the discs are square, oval, elliptical, star shaped, rectangular, trapezoidal, polygonal, Reuleaux polygonal, circularly indented polygonal, lobed, scalloped shaped, kidney shaped, wedge shaped, symmetrical, asymmetrical, or have any other suitable shape.

In some implementations, the housing includes one or more lids or other openings. Indeed, in some cases, the housing includes a lid, which in some cases, is removable, closeable, or openable. In some cases, the manipulation discs are interchangeable, allowing for discs with different stiffnesses, curvatures, thicknesses, or other characteristics to be placed within the housing. In some instances, this configuration allows for different numbers of discs to be placed within the housing. Accordingly, in some cases, a single instrument is useful for measuring multiple different ranges of manipulation forces.

In some implementations, the housing includes a rigid material. In some implementations, however, the housing includes a pliable, flexible, or resilient material. Indeed, in some embodiments, the housing includes a pliable or flexible material that allows for the housing to be freely or readily compressed. Thus, in some iterations, the manipulation discs provide substantially greater resistance to manipulation than the housing.

Some implementations of the manipulation instrument include a flexible or resilient element, such as a manipulation disc, an arm, or any other suitable flexible or resilient component. In some cases, the flexible or resilient element is configured to deflect when a spinal manipulation force is applied to the spine of the patient (e.g., in some cases, a manipulation disc is configured to flatten or become flatter, a rod is configured to bend, or another component is configured to otherwise deform, deflect, or change to another configuration). In some implementations, the gauge is configured to measure the applied spinal manipulation force based on the deflection of the flexible or resilient element. In some cases, the flexible or resilient member is flexible but not resilient, such that when the member is deformed, it maintains its shape to allow for a manipulation force to be easily measured, even after the manipulation instrument has been removed from the patient. In some cases, however, the flexible or resilient member is both flexible and resilient such that the manipulation instrument can be reused one or more times (e.g., many times following autoclaving).

To illustrate the foregoing, in some implementations, the flexible or resilient element includes a resilient arm that is configured to deflect when the spinal manipulation force is applied, and the gauge includes an indicator configured to measure the deflection of the arm. In some cases, the arm includes a first or a second arm of a manipulator. In some cases, the indicator includes one or more needles (or one or more rods, shafts, bars, arrows, projections, dials, indicators, or any other suitable physical component capable of indicating a measurement based on its position). The needle, in some implementations, is coupled to the first or second arm (in some cases, at or near the tip of the applicable arm). In some implementations, the needle is coupled to the applicable arm at a single location, so that when the arm is deflected or flexed, the needle remains in an unflexed configuration.

In some instances, the instrument includes one or more indicia. Although the indicia can include any indications of any kind that may aid in deciphering a measured value, in some cases, the indicia includes a scale having one or more markings that correspond to certain amounts of force applied to the arm. For example, in some cases in which a specific amount of force (e.g., 5 newtons, 150 newtons, or any other specified amount of force) is applied to the arm, the arm is deflected a certain distance, but the needle remains undeflected. Thus, in some cases, the distance between the initial position of the arm and its deflected position can be precisely determined through reference to the position of the needle relative to the arm. In some cases, the indicia can greatly aid a human being in making this determination (e.g., by including one or more markings that the needle points at when the arm is deflected with 1, 5, 10, 20, 50, 100, 150, 200, or 250 newtons of force, or any subrange of the foregoing).

In some implementations, the instrument includes one or more indicia where the flexible or resilient component includes the manipulation disc. For example, in some cases, the indicator is configured to measure a change in height of the stack of manipulation discs. Some iterations of the indicator include one or more indicia that correspond to a height of the stack at different manipulation loads.

In some implementations, the gauge includes or is otherwise configured to function with an air pressure sensor or any other suitable component for measuring a spinal manipulation force applied to the instrument. In some implementations, when a desired spinal manipulation force is applied using the instrument (as determined by the gauge), a spinal implant of the proper size (e.g., the size that would apply the desired amount of manipulation force) is inserted into the space between two vertebrae of the spine where the manipulation force was measured.

Some implementations of the systems and methods disclosed herein include a method for applying and measuring a spinal manipulation force. In some cases, the method (which can be varied in any suitable manner) includes one or more of the following: obtaining one or more manipulation instruments for applying one or more spinal manipulation forces; obtaining one or more gauges for measuring the spinal manipulation force; applying the spinal manipulation force with the instrument; and measuring the spinal manipulation force with the gauge.

In some implementations, the applying the spinal manipulation force with the instrument includes gradually increasing an application of the spinal manipulation force until the spinal manipulation force reaches a desired value, as measured with the gauge.

In some implementations, the desired value is anywhere between 1 newton and 350 newtons (or any subrange thereof). For example, in some implementations, the desired value is approximately between 50 newtons and 250 newtons, and in some implementations the desired value is between 75 newtons and 150 newtons.

In some implementations, the desired value of applied force is related to the contact area between the instrument and the bone. For example, in some implementations, the desired value is approximately 8 newtons ± 4 newtons per square millimeter of contact area between the instrument and the bone.

In some implementations, the method includes measuring a distance between two spinal vertebrae when the spinal manipulation force reaches the desired value. In some cases, the measuring a distance is done with a tool separate from the one used to apply the manipulation, but, in some cases, the same tool is used for applying the manipulation and measuring the distance (and, in some cases, measuring the manipulation force as well). In some cases, the method includes inserting an implant having a thickness approximately equal to the distance as measured.

Some implementations of the disclosed systems and methods include a system for applying a desired amount of manipulation force to a patient’s spine. In some instances, the system includes an instrument configured to apply a trial force to a pair of vertebrae of the patient’s spine. While the instrument may be any suitable instrument (as discussed herein), in some implementations, the instrument includes one or more of a flexible or resilient element configured to deflect upon application of the trial force and a gauge for measuring the trial force based on a degree of deflection of the flexible or resilient element. In some implementations, the system includes a spinal implant configured to apply the desired amount of manipulation force to the patient’s spine, in accordance with the trial force as measured. In some cases, the system includes a plurality of spinal implants having differing characteristics (e.g., different thicknesses, different materials, different stiffnesses, etc.), thereby allowing a healthcare worker to select a suitable spinal implant having the desired characteristics (or to vary the procedure) based on an applied manipulation force determined to be suitable through measurement of the force or based on a distance between vertebrae when a set amount of force is provided to the vertebrae, or when a set force is provided to a known contact area of the vertebrae.

The systems and methods disclosed herein may be combined with one another in any suitable manner, and any element or portion of any disclosed system or method may be combined with any other element or portion of any disclosed system or method, unless expressly stated otherwise herein. Additional implementations are discussed herein, and additional uses and features will be apparent to those of skill in the art based on this disclosure.

### BRIEF DESCRIPTION OF THE DRAWINGS

The objects and features of the present disclosure will become more fully apparent from the following description and appended claims, taken in conjunction with the accompanying drawings. Understanding that these drawings depict only some typical embodiments of the disclosed systems and methods and are, therefore, not to be considered limiting of its scope, the systems and methods will be described and explained with additional specificity and detail through the use of the accompanying drawings in which:

FIG. 1 shows a plan view of an instrument for applying a spinal manipulation force and measuring an associated characteristic, in accordance with some embodiments of the described systems and methods;

FIGS. 2A-2B show perspective views of various configurations of an instrument, according to some embodiments;

FIGS. 3A-3B show perspective views of various configurations of an instrument, according to some embodiments;

FIGS. 4A-4B show perspective views of an instrument, in accordance with some embodiments;

FIGS. 5A-5C show elevation views of various instruments, in accordance with some embodiments;

FIG. 6 shows a perspective, cross-sectional view of an instrument, in accordance with some embodiments;

FIG. 7A shows a perspective, cross-sectional view of an instrument, in accordance with some embodiments;

FIG. 7B shows an elevation, cross-sectional view of portions of an instrument, in accordance with some embodiments;

FIG. 7C shows a perspective view of portions of an instrument, in accordance with some embodiments;

FIG. 8 shows a gauge of an instrument, in accordance with some embodiments; and

FIG. 9A-9C show various views of an instrument, according to some embodiments.

### DETAILED DESCRIPTION

Systems and methods for measuring and applying a spinal manipulation force (e.g., a compressive force, a distractive force, or another force) are provided. Some implementations of the systems and methods include an instrument having one or more manipulators or gauges. In some cases, the manipulator is configured to apply the spinal manipulation force to a spine of a patient. In some cases, the gauge is configured to measure the spinal manipulation force. In some cases, the gauge is configured to measure a resultant displacement (e.g., a distance between a first vertebra and a second vertebra as a result of application of the manipulation force). In some cases, the instrument is configured to apply a known amount of force. Thus, in some cases, a practitioner can ascertain the appropriate size for an implant by knowing the distance between vertebrae as a result of an appropriate application of force.

A description of embodiments will now be given with reference to the FIGS. It is expected that the present systems and methods may take many other forms and shapes. Hence the following disclosure is intended to be illustrative and not limiting, and the scope of the disclosure should be determined by reference to the appended claims.

Some embodiments of the disclosed systems and methods include one or more manipulators configured to apply a spinal manipulation force. The term “manipulation” as used herein may refer to spinal or vertebral compression, spinal or vertebral distraction, and other forces operating on the spine, either as a whole or on one or more individual vertebrae (or other parts of the spine), unless the context clearly indicates otherwise.

Although the manipulator can include any component configured to apply a spinal manipulation force, various embodiments of manipulators are disclosed herein. Likewise, some embodiments of the disclosed systems and methods include one or more gauges. Although the gauge can include any suitable component configured to measure a spinal manipulation force or displacement of vertebrae (or any other suitable objects), various embodiments of gauges are disclosed herein. In some embodiments, the manipulator includes one or more flexible or resilient elements configured to deflect when the spinal manipulation force is applied. In some embodiments, the gauge measures the spinal manipulation force based on a deflection of the flexible of resilient elements. In some embodiments, the manipulator applies a set force after which a distance (e.g., between vertebrae or other desired objects) may be measured. Accordingly, in some embodiments, the spinal manipulation force can be correlated to a separation distance of two or more vertebrae of the spine, thus allowing for a spinal implant of a suitable size (and configured to apply the desired amount of manipulation) to be selected and inserted into the patient’s spine. As a wide variety of systems and methods may be used to accomplish these purposes, various embodiments are disclosed below.

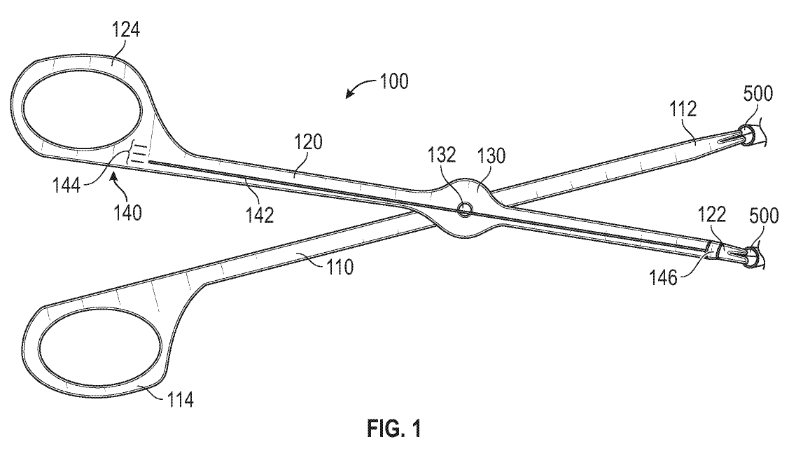

As shown in FIGS. 1-5C, some embodiments of the disclosed systems and methods include a manipulator 100. As shown in FIGS. 1-4B, some embodiments of the manipulator 100 include one or more arms, such as a first arm 110 and a second arm 120 (and any number of additional arms). Although the arms may be any size and shape, and may include any suitable component that is useful for medical procedures or that can otherwise be associated with medical instruments, some embodiments of the manipulation instrument generally resemble a pair of calipers, scissors, pliers, reverse pliers, forceps, hemostats, rongeurs, shears, clippers, grippers, or any other two-armed tool. Notwithstanding the foregoing, some embodiments have more than two arms, only one arm, or no arms at all.

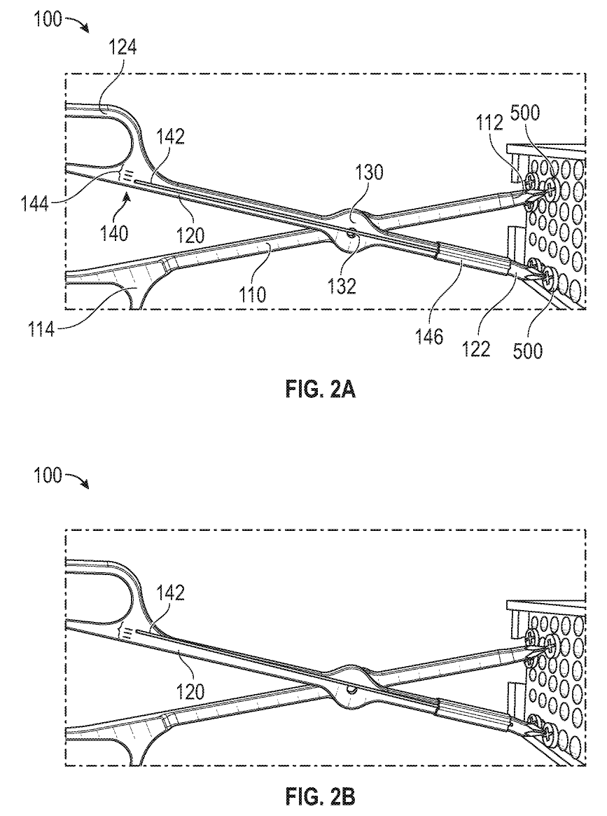

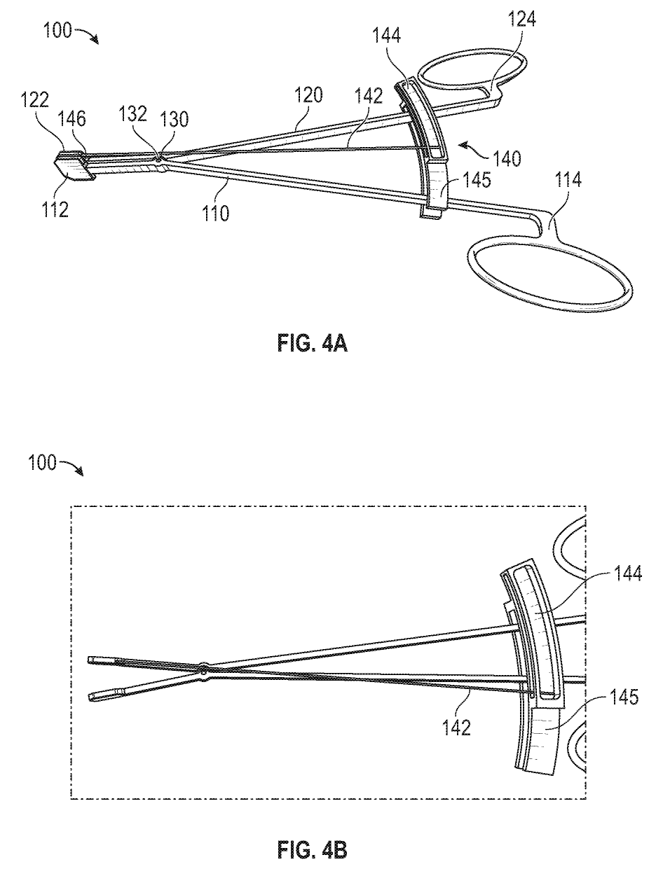

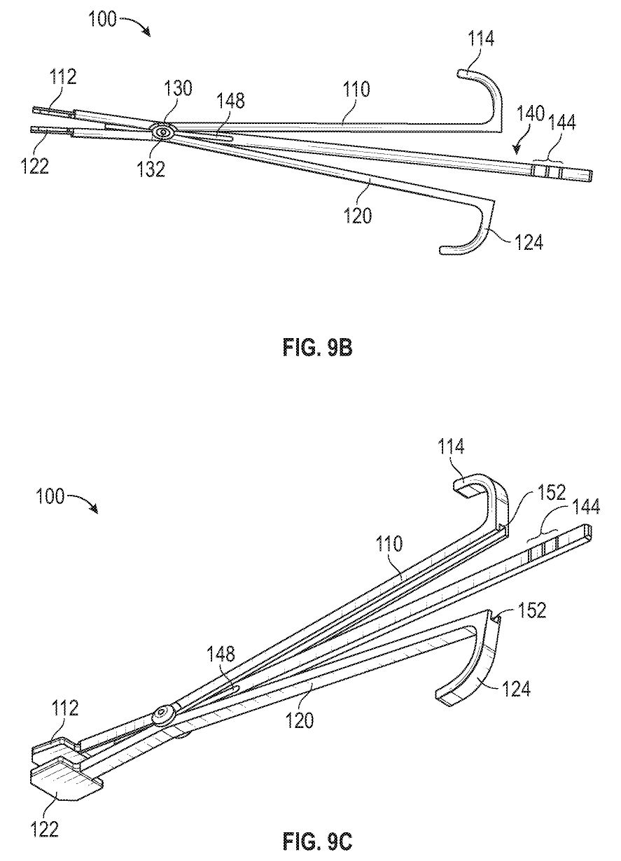

In some embodiments one or more of the arms includes one or more tips. As an example, in some embodiments, the first arm includes a first tip 112. In some embodiments, the second arm includes a second tip 122. Although the tips may include any feature of a tip used in connection with any tool or medical instrument (e.g., comprising a point, a drive feature, a plate, a planar portion, a duck bill, a blade, a coupler, a catch, or any other suitable feature), in some embodiments, at least one of the first tip 112 and the second tip 122 is configured to selectively couple to a spinal implant 500. For example, as shown in FIGS. 1-2B, in some embodiments, the tip 112, 122 is shaped and sized (e.g., comprises: a rounded end, a catch, a hook, a coupler, a projection, a screw head projection, a drive, a frictional engagement, a mechanical engagement, or any other suitable feature that is configured) to correspond to an anchor (e.g., a screw, nail, bolt, rivet, shaft, plate, pin, wire, implant 500, or any other component configured to be attached to a patient’s spine). In some embodiments, as shown (for example) in FIGS. 4A-4B and 9A-9C, the tip 112, 122 is configured to contact, couple directly to, or otherwise interface with the spine itself (e.g., by having a surface sized and shaped appropriately to contact a surface of a vertebra, being sized and shaped like a spinal implant, having a blade or planar portion, or otherwise being configured to interface with the spine).

Returning to FIGS. 1-4B, in some embodiments, one or more of the arms 110, 120 include one or more handles 114, 124. For example, in some cases, the first arm 110 includes a first handle 114. In some cases, the second arm 120 includes a second handle 124. Although one or both handles can include any feature useful for gripping the associated arm (either directly or through the intermediate use of a tool), such as one or more grips, protrusions, indentations, bars, slots, rings, texturized surfaces, or other gripping components, some embodiments of a handle include a ring. In some cases, the ring is configured to receive all or part of a thumb, hand, or one or more fingers of a user (e.g., a physician). In some embodiments, the handles are configured to allow a practitioner to selectively bias the arms away from each other or toward each other (in some cases, using one hand).

In accordance with the foregoing, some embodiments of the manipulator 100 are configured such that applying a force to at least one of the first handle 114 and the second handle 124 causes at least one of the first tip 112 and the second tip 122 to apply the spinal manipulation force to the spine (e.g., when the tips are engaged, directly or indirectly, with the spine).

Some embodiments of the manipulator 100 include one or more pivots 130. For example, in some cases, the first arm 110 and the second arm 120 are coupled together to form the pivot 130. Some embodiments include a pivot coupler 132 (e.g., a screw, bolt, nail, rod, thread, hinge, articulation, axis, ball-and socket joint, cylindrical joint, joint, bearing, or any other type of fastener that could be used to couple multiple objects (e.g., arms) together to form a fulcrum, pivot, hinge, axle, spindle, or other static or dynamic connection point).

As shown in FIGS. 1-2B, in some embodiments, the first arm 110 is coupled to the second arm 120 (e.g., via the pivot 130) such that when the first arm is biased toward the second arm (e.g., when a user squeezes the handles 114, 124 toward each other), the first tip 112 is biased toward the second tip 122 (e.g., the tips come together, as with traditional scissors or pliers). In some such embodiments, squeezing the handles of the manipulator 100 (or otherwise biasing one handle toward the other or both handles toward each other) causes the first and second tips, when coupled to spinal vertebrae, to place a compressive force on one or more of the spinal vertebrae in question.

As shown in FIGS. 4A-4B and 9B-9C, some embodiments of the manipulator 100 include a configuration where the first arm 110 is coupled to the second arm 120 (e.g., via the pivot 130) such that when the first arm is biased toward the second arm, the first tip 112 is biased away from the second tip 122 (e.g., the tips diverge, as with reverse pliers). In some such embodiments, squeezing the handles of the manipulator 100 (or otherwise biasing one handle toward the other or both handles toward each other) causes the first and second tips to separate from one another, thereby placing a distractive force on one or more spinal vertebrae that may be coupled to or located proximate to one of the tips.

In some embodiments, the first arm 110 and the second arm 120 are naturally biased towards or away from each other (e.g., via one or more springs, elastics, or any other suitable resilient member disposed between or coupled to the arms, a tension rod, or any other suitable biasing mechanism) in order to maintain the manipulator 100 in a closed or open configuration (as desired) when the manipulator is at rest.

In some embodiments, the manipulator 100 comprises one or more plastics, polymers, metals, fiberglass, or other materials that that allow the manipulator to be readily disposed. In some embodiments, however, the manipulator is configured to be used multiple times. Although the manipulator can include any material useful for constructing a medical device, including one or more types of metal, metal alloy, ceramic, plastic, polymer, fiberglass, glass, or any other suitable material, some embodiments are formed of heat-resistant materials (e.g., metal) that can be placed into a sterilizing autoclave without being damaged or permanently deformed. Indeed, some embodiments lack one or more components that are not typically autoclave-safe (e.g., certain plastics, electronics, or other meltable or temperature-sensitive materials). In some embodiments, one or more components of the manipulator (e.g., one or more arms 110, 120) are formed of or otherwise comprise one or more flexible or resilient materials configured to deflect when the spinal manipulation force is applied (as discussed more fully below).

The arms 110, 120 (and any other component of the manipulator 100) may be any suitable length and thickness, and may include any suitable cross-sectional shape (e.g., circle, square, triangle, and any other regular or irregular shape) and have any other desirable characteristic that allows the manipulator to function as described herein.

According to some embodiments, the manipulator 100 is configured to apply a predetermined amount of force when actuated. For example, in some cases, the manipulator is configured to apply a predetermined amount of force when the arms are biased together such that they (or one or more other contacts) touch. The predetermined amount of force can be any suitable amount, such as anywhere between 1 newton and 1,000 newtons (or any subrange thereof). For example, in some embodiments, the predetermined value is approximately between 50 and 250 newtons, and, in some embodiments, the desired value is between approximately 75 newtons and approximately 150 newtons. Indeed, in some embodiments, the predetermined value is approximately 100 newtons ± 10 newtons and, in some embodiments, the desired value is between 45 newtons and 160 newtons. In some other cases, such as for uses in the lumbar spine, the force can be larger, such as 400 newtons ± 20 newtons, 300 newtons ± 15 newtons, or between 190 newtons and 210 newtons. Also, while the force is not correlated to the contact area between the bone and the instrument in accordance with some embodiments, in other embodiments, the force is correlated to the contact area between the bone and the instrument. For example, in some embodiments, the predetermined value exerted by the manipulator is anywhere between 5 newtons and 25 newtons (e.g., between 8 newtons and 20 newtons, or 8 newtons ± 4 newtons) per square millimeter of contact area between the bone and the instrument.

In some embodiments configured to provide a predetermined amount of force, the predetermined amount of force is adjustable. In this regard, the manipulator 100 can be adjusted in any suitable manner that allows the predetermined amount of force to be adjusted (e.g., by changing a length of one or both of the arms 110, 120, adding or more removing one or more braces or reinforcements, adjusting a position of one or more contacts, adjusting arm movement limits, or in any other suitable manner). For example, some embodiments include arms having one or more adjustable characteristics, such as an adjustable length, adjustable cross section, reinforcement members configured to be selectively coupled to the arms, or any other components configured to adjust the predetermined amount of force. Indeed, in some embodiments, one or more of the arms have an adjustable length or contact surfaces are configured to be selectively moved. In this regard, such adjustments can be done and selectively maintained in any suitable manner (i.e., via one or more clamps, ratcheting mechanisms, detents, cams, catches, mechanical engagements, frictional engagements, or in any other suitable manner).

In some embodiments, multiple instruments are provided together in a kit, with different instances of such instruments being configured to apply different predetermined amounts of force. For example, some embodiments include a 50 newton instrument, a 100 newton instrument, a 150 newton instrument, and a 200 newton instrument (in each case, the instrument being configured (or modified) to apply a force within 1%-20% of the stated value (or any subrange thereof)). In some cases, the instruments are color-coded, labeled, or otherwise differentiated from each other to allow a practitioner to easily select the instrument configured to apply the proper amount of force.

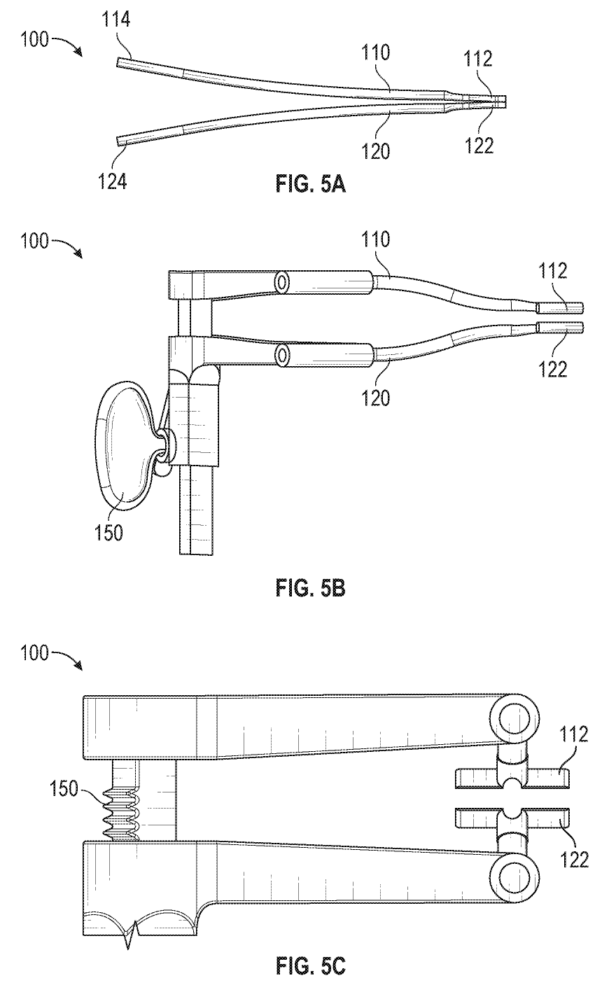

The manipulator 100 can have any other suitable configuration as well. For example, while some embodiments of the manipulator include handles 114, 124 for biasing the tips 112, 122 together or apart, some embodiments include other mechanisms for doing so, such as one or more actuators 150 configured to move the tips together or apart. Where an actuator is included, the actuator can include any component for moving the tips together or apart, such as one or more motors (stepper motors, servomotors, bushed or brushless motors, induction motors, or any other motors), hydraulic actuators, pneumatic actuators, electric actuators, linear actuators, piezoelectric actuators, thermal actuators, electromagnetic actuators, worm gears, rack-and-pinion mechanisms, ratchets, gears, or any other suitable types of actuator. By way of non-limiting illustration, FIGS. 5A-C show additional configurations for manipulators 100 that can be outfitted with additional components as discussed herein or otherwise adapted to form a manipulation instrument in accordance with this disclosure. For example, FIG. 5A shows some embodiments of a manipulator 100 with a first arm 110 and a second arm 120, wherein a first tip 112 of the first arm 110 and a second tip 122 of the second arm 120 are configured to diverge as a first handle 114 is moved toward a second handle 124, as the first arm 110 and the second arm 120 are configured to act as levers with respect to each other due to each having a curved shape such that the tips are pried apart due to each arm having a point acting as a fulcrum for the other. FIG. 5B shows some embodiments of a manipulator 100 which, instead of being configured to separate the first tip 112 and the second tip 122 by squeezing a handle, is instead configured to allow for actuation of the first tip 112 and the second tip 122 toward or away from each other by means of an actuator 150 configured to move at least one of the first arm 110 and the second arm 120 toward or away from the other. Similarly, FIG. 5C shows some embodiments of a manipulator 100 configured to apply a compressive force, wherein the first tip 112 and the second tip 122 are configured to be biased away from or toward one another via an actuator 150. In any case, one or more gauges 140 (of any suitable kind, as discussed herein) can be coupled to or inserted between the arms, or otherwise configured for use in connection with the manipulator.

As shown in FIGS. 1-4B, some embodiments of the described systems and methods include one or more gauges 140. The gauge can include any component for taking one or more measurements or conveying such measurements to a user. For example, in some embodiments, the gauge is configured to measure the manipulation force or convey the value of the manipulation force to the user. In some embodiments, the gauge is configured to measure a distance (e.g., a size of a disc space or distance between vertebrae, or a change in such brought about by application of the manipulation force) or to convey the value of the distance to the user. By way of non-limiting illustration, FIGS. 9A-C show some embodiments of a manipulator 100 having a gauge 140 configured to measure a size of a disc space when a known amount of force is applied using the manipulator.

Some embodiments of the gauge 140 include one or more indicators 142 configured to convey a measurement to a user. The indicator can include any component suitable for conveying such a measurement, such as one or more needles, rods, shafts, bars, projections, arrows, sliders, dials, lasers, gradations, markings, meters, displays, lights, pressure indicators, or other components configured to dynamically shift (e.g., change position, orientation, size, color, or any other perceivable characteristics) to display, emit, or otherwise convey one or more measurement values.

In some embodiments, the gauge 140 measures the manipulation force, the distance between vertebrae, or any other suitable characteristic based on the deflection of one or more flexible or resilient elements. In some embodiments, the flexible or resilient element includes an arm (e.g., one or more of the first arm 110, the second arm 120, or additional arms) that is configured to deflect (e.g., bend, bow, flatten, or otherwise flex) when the spinal manipulation force is applied. In this regard, some embodiments of the flexible or resilient element include a rod or another component with a known stiffness, such that the manipulation force can be determined based on the deflection distance.

In some embodiments, the indicator 142 is configured to measure the deflection of one or more flexible or resilient components. For example, in some cases, the indicator includes one or more needles (or rods, shafts, arrows, projections, bars, dials, lasers, plungers, sensors, or other components capable of indicating a measurement based on its position).

The indicator 142, in some embodiments, is coupled to any suitable portion of the manipulator 100 (e.g., at or adjacent to: a tip 112, 122; a joint 130; an arm 110, 120; or any other suitable portion). Indeed, in some embodiments, the indicator is coupled to a non-resilient, rigid, or substantially rigid portion of the manipulator (e.g., a rigid tip 112, 122 of the manipulator). In some embodiments, however, the indicator is coupled to a flexible or resilient material used in the manipulator (e.g., a flexible or resilient tip or arm).

While the indicator 142 can be coupled to the manipulator 100 (e.g., an arm 110, 120; a tip 112, 122; pivot 130, or any other suitable portion) in any suitable manner, in some embodiments, the indicator is coupled to the manipulator at (or only at) a single location, so that when a flexible or resilient material or portion of the manipulator is deflected (e.g., one or more arms are deflected), the indicator remains in an undeflected configuration.

In some embodiments, the indicator 142 is coupled to the rigid, flexible, resilient, or any other suitable portion of the manipulator 100 in any suitable manner, including via one or more connectors 146. While the connector can include any suitable feature that couples the indicator to a portion of the manipulator, in some cases, the connector includes one or more sleeves, welds, rivets, joints, screws, nails, bolts, staples, frictional engagements, mechanical engagements, magnets, clamps, bands, adhesives, ties, integral connections, fasteners, or other coupling mechanisms. Where the indicator is coupled to the manipulator and where at least a portion of one or both of the arms 110, 120 comprise a flexible or resilient material, in some embodiments, the indicator is coupled to the manipulator at or near the tip 112, 122 of an arm. In any case, some embodiments of the indicator are coupled to the tip within 0.5 mm, 1 mm, 2 mm, 3 mm, 5 mm, 10 mm, or 25 mm (or any subrange between 0.1 mm and 25 mm, or any other suitable distance) of the end that is configured to apply the manipulation force. Indeed, in some cases a more noticeable measurement may be obtained using a smaller indicator where the indicator is coupled in a specific position (e.g., as close to the force-applying end of the manipulator as possible, at the pivot, or in another desired location). By way of non-limiting illustration, FIG. 1 shows a manipulator 100 having a gauge 140 comprising an indicator 142 substantially in the form of a needle that is coupled to an arm 120 via a connector 146 in the form of a band such that when a resilient material or portion of the arm 120 deflects (in some cases, a portion of the arm that is between the pivot 130 and the handle 114, 124), the indicator 142 shows the amount of deflection relative to the undeflected indicator 142.

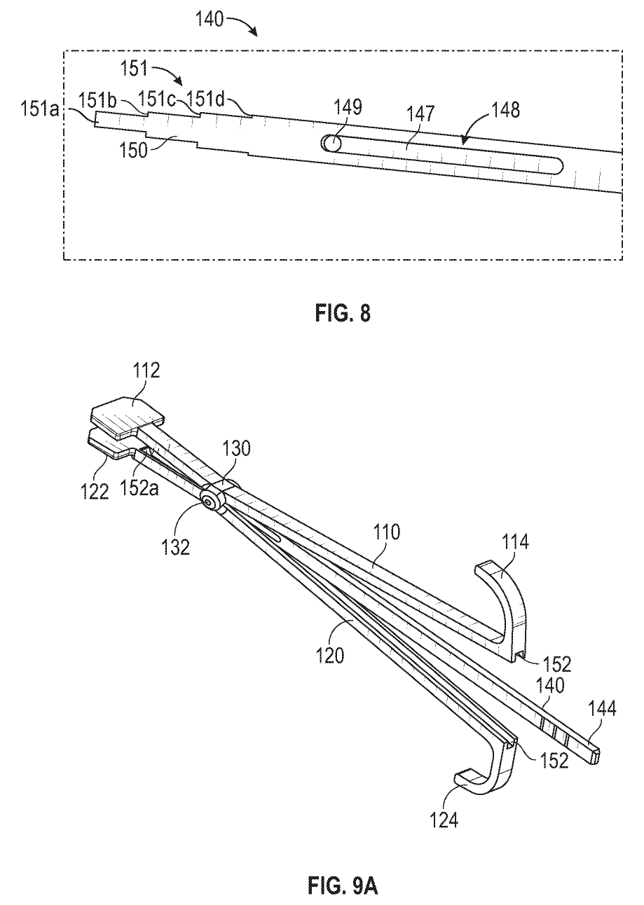

In some embodiments, the gauge 140 includes one or more interfaces 150 configured to interface with one or more portions of the manipulator 100. In particular, some embodiments of the interface cause the gauge to interact with the manipulator differently depending on a position of the manipulator, an amount of deflection of the manipulator, or another characteristic of the manipulator. For example, some embodiments of the interface include one or more stepped, sloped, tapered, wedge-shaped, angled, or graduated surfaces, thereby allowing larger portions of the gauge to pass through a larger gap. In some cases, the interface comprises one or more stepped surfaces. In some such cases, the stepped surfaces include a first portion having a first measurement (e.g., diameter, width, height, or other measurement), a second portion having a second measurement, and (in some cases) any additional number of portions having additional measurements (e.g., a third portion having a third measurement, a fourth portion having a fourth measurement, a fifth portion having a fifth measurement, etc.). By way of non-limiting illustration, FIG. 8 shows a gauge 140 having an interface 150 in the form of a series of stepped surfaces. As shown in FIGS. 9A-9C, this allows the interface 150 to pass into a space between the first tip 112 and the second tip 122 of the manipulator, being able to move farther where the space between the tips is larger, and being stopped sooner due to the mechanical interference of the stepped surfaces against one or more portions of the manipulator (such as the tips 112, 122, or an end of a groove 152 as discussed in more detail below).

In some embodiments, the gauge 140 includes one or more indicia 144. The indicia can include any suitable type of scales, markings, gradations, protrusions, grooves, notches, slots, colors, spectrums of color, or other indications of any kind that may aid a human in deciphering a measured value. Indeed, in some cases, the indicia include one or more scales having markings that correspond to certain values of the manipulation force. In some cases, when a specific amount of force (e.g., 5 newtons, 150 newtons, or any other specified amount of force) is applied to the arm 110, 120, the arm is deflected a certain distance, but the indicator 142 remains undeflected (e.g., as shown in FIG. 2B). Thus, in some cases, the distance between the initial position of the arm 110, 120 and its deflected position can be precisely determined through reference to the position of the indicator 142 relative to the arm. In some cases, the indicia 144 show a position of the arm relative to the needle. In some cases, the indicia include one or more markings for an initial position of the arm, a target position (or range) of the arm (for example, a position at which a desired manipulation force is applied by the arm), a warning position (e.g., where too much force is being applied), or any other useful position. Accordingly, in some embodiments, the indicia may greatly aid in making the determination of how much force is being applied, based on the deflection of the flexible or resilient component, as shown based on the relative position of the indicator (e.g., by including one or more markings to which the indicator points when the arm is deflected with up to 1, 5, 10, 20, 50, 100, 150, 200, 250, or 300 newtons of force, or any subrange thereof, or any other suitable amount of force). In some embodiments, the indicia include markings delineating an optimal manipulation force or an optimal range of manipulation force. In some cases, the indicia include markings in set force increments, and in some cases, the indicia include markings based on desired measurements or historic results.

While placement of the indicia on the instrument can be determined in any suitable manner (e.g., through calculation based on the known rigidity or any other suitable characteristic of one or more portions of instrument or in any other suitable manner), in some cases, a known amount of force is applied to one or more handles of the instrument and corresponding markings that align the needle with such known forces are placed on or are otherwise associated with the device.

Some embodiments include multiple sets of indicia. For example, as shown in FIGS. 4A and 4B, some embodiments include an alternative set of indicia 145 in addition to (or instead of) the indicia 144. These multiple sets of indicia may indicate different measurements. For example, in some embodiments, one set measures load (or manipulation force) and one set measures displacement or distance.

To provide some specific illustrative examples (in accordance with some embodiments as described above), FIGS. 2A and 2B show an instrument having a manipulator 100 with a gauge 140. In these illustrations, the manipulator includes a first arm 110 coupled to a second arm 120 via a pivot coupler 132 of a pivot 130. Additionally, a first tip 112 and a second tip 122 are connected to an implant 500 (e.g., anchors configured to be attached to vertebrae of a patient). The gauge includes an indicator 142, which, in the case of these illustrations, is substantially in the shape of a needle. The indicator in these illustrations is coupled to the second arm 120 via a connector 146. Moreover, in these illustrations, the indicator points to indicia 144. In FIG. 2A, the indicator is pointing to a medial marking, as the instrument is at rest (e.g., no manipulation force, or very little manipulation force, is currently being applied). In FIG. 2B, a manipulation force is applied by squeezing a first handle 114 and a second handle 124, thereby biasing the first and second arms toward each other. As the arms are biased toward each other, the second arm 120 deflects slightly. Since, in this shown embodiments, the indicator 142 is only fixed to the second arm 120 near the tip 122, the indicator does not deflect along with the arm. Thus, the indicator in FIG. 2B now points to a position more lateral on the instrument than the resting position—as seen by the fact that the indicator now points to a more lateral marking.

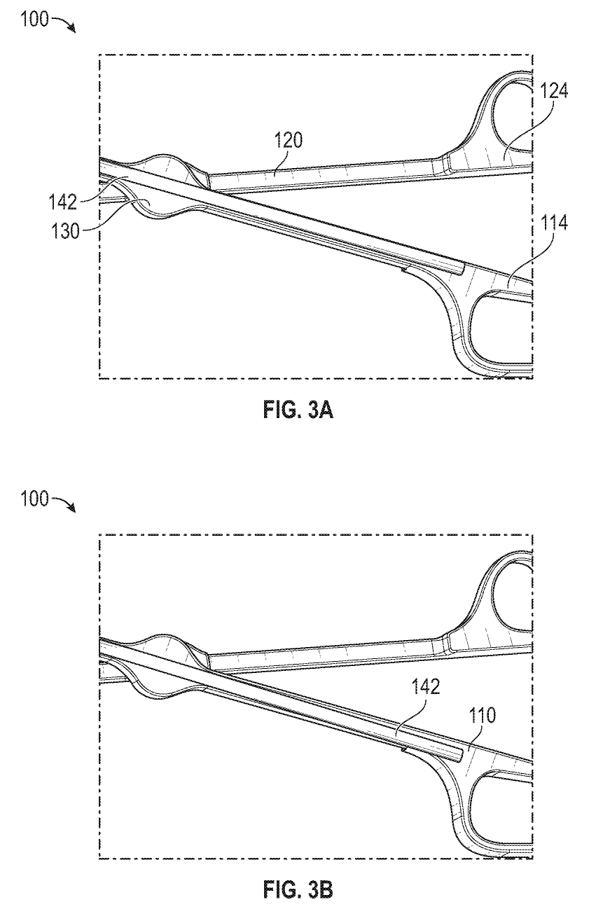

FIGS. 3A and 3B show examples in accordance with similar embodiments to those seen in FIGS. 2A and 2B. In this regard, FIG. 3A shows that in some embodiments an indicator 142 that is flush with a first arm (110, as shown in FIG. 3B), while a manipulation force is not applied. When a manipulation force is applied, as shown in FIG. 3B (by squeezing handles 114, 124), the first arm 110 is deflected while the indicator remains straight, resulting in a noticeable offset between the indicator 142 and the first arm 110.

FIGS. 4A and 4B show additional examples, in accordance with some embodiments. In these illustrations, the instrument is configured such that when a practitioner squeezes the handles 114, 124, the tips 112, 122 are biased away from each other as opposed to toward each other, due to the particular configuration of the pivot 130. Note that in both FIG. 4A and FIG. 4B, no manipulation force is applied, even though FIG. 4A shows the manipulator 100 in a closed configuration, and FIG. 4B shows the manipulator in an open configuration. As no force is applied, the indicator 142 points to essentially the same location on the indicia 144 in both FIGS. (indicating that the load, or manipulation force, is essentially zero). However, the position of the secondary indicia 145 is shifted with respect to the indicator 142, because the secondary indicia includes a displacement (e.g., measuring distance between vertebrae or a change in such distance) scale. In short, a wide variety of configurations for the gauge 140 are possible, including many indicators that provide measurements of force, displacement, or other characteristics based on the deflection, position, or other characteristics of one or more flexible or resilient elements.

As mentioned previously, some embodiments include one or more features for measuring distance (e.g., a height of an intervertebral disc space when a spinal manipulation force—such as one having a known or measured value—is applied) or displacement (e.g., a change in such height). For example, some embodiments are configured to allow a physician to apply a set force (either having a known value, or including a way for the physician to discern the value, such as by measuring and displaying the force to the physician in real time). By way of non-limiting illustration, FIGS. 8 and 9A-C show some embodiments that may be particularly suitable for measuring the distance between vertebrae created as a result of application of a known amount of force. Thus, the known force can be applied in an amount that is safe (e.g., unlikely to damage the bone, the spine, the ligaments of the spine, or another portion of the patient), and a proper implant size can be determined as a result of the applied force.

As discussed above, some embodiments include one or more manipulators 100 and one or more gauges 140. In some embodiments, the manipulator is configured to apply a specific, known amount of force. For example, in some cases, by pressing the first handle 114 down to the second handle 124 (e.g., all the way, so that the two handles (or any other desired contacts) touch, or any suitable portion of the distance between the handles), the instrument (based on the length of the first arm 110 and the second arm 120, the stiffness of the arms, the shape of the arms, and any other applicable characteristics of the instrument) is configured to apply a specific amount of force, such as any amount between about 4 newtons and 450 newtons, including any subrange thereof (e.g., between 50 newtons and 150 newtons, between 300 newtons and 400 newtons, or any other subrange). Indeed, in some embodiments, the manipulator is configured to apply about 50 newtons ± 5 newtons, 100 newtons ± 10 newtons, 150 newtons ± 15 newtons, 350 newtons ± 35 newtons, 400 newtons ± 40 newtons, 450 newtons ± 45 newtons, or any other suitable amount. Also, the applied force can be related to the contact area. For example, in some embodiments, the applied force is configured to be between 8 newtons and 20 newtons per square millimeter of contact area between the instrument and bone (or other contact surface).

According to some embodiments, a separate instrument is provided for each desired amount of force. Accordingly, some embodiments include a kit of instruments, with the various instruments corresponding to different amounts of force. In some cases, the instruments are color-coded, labeled, or otherwise configured to readily convey to a practitioner the amount of force the instrument is configured to apply. The foregoing notwithstanding, some embodiments of the manipulator 100 are adjustable to provide different amounts of force (including any of the amounts discussed above). The manipulator can be adjustable in any suitable manner, such as by having one or more adjustable arms, interchangeable stiffening members, variable diameters, adjustable pivots 130, adjustable contacts, movement limiting mechanisms, or in any other suitable manner. For example, in some embodiments, one or more of the first arm 110 and the second arm 120 has an adjustable length (e.g., via telescopic adjustment, one or more arm extensions, a slidable arm that is configured to be locked at different positions, or otherwise). Indeed, in accordance with some embodiments, the first arm 110 and the second arm 120 are configured or altered such that one or both of their lengths is related to their corresponding cross section so as to decrease the force applied by the practitioner to one or both arms of instrument without decreasing the output manipulation force at the first tip 112 or the second tip 122.

Although the gauge 140 can be coupled to or otherwise be associated with the manipulator 100 in any suitable manner (e.g., via one or more connectors 146, as discussed previously), some embodiments of the gauge are configured to be disposed between the first arm 110 and the second arm 120 of the manipulator. Although this can be done in any suitable manner, some embodiments of the manipulator include one or more recesses 152 in which the gauge is configured to be disposed.

Where one or more recesses 152 are included, the recesses can have any suitable configuration allowing them to house all or part of the gauge 140. For example, some embodiments include one or more slots, grooves, notches, passages, or other recesses extending along a length or at least a portion of one or more of the first arm 110 and the second arm 120. While the recesses can be included on (or in) any suitable portion of the manipulator 100 (e.g., on a lateral portion, an exterior portion, through one or more of the arms, at or through the pivot, or anywhere else), some embodiments of the recesses are defined by an inner surface of one or more of the first arm and the second arm (e.g., the surfaces that are facing each other), thereby allowing the gauge to be disposed between the first arm and the second arm and in the recess formed on the arm on either side. In some cases, the recesses are sufficiently deep as to allow the gauge to be disposed between the first arm and the second arm when the first arm and the second arm are biased all the way toward each other such that they (or one or more other contacts) come into contact with each other (without the gauge preventing the arms from touching). By way of non-limiting illustration, FIGS. 9A-9C show some embodiments of manipulators 100 having gauges 140, wherein the gauges 140 are disposed between a first arm 110 and a second arm 120 of the manipulators 100, and wherein a recess 152 in the form of a slot or groove is formed in each of the first arm 110 and the second arm 120, the recess 152 in the first arm 110 being configured to house half (or any other suitable portion of) the gauge 140, and the recess 152 in the second arm 120 being configured to house the other half (or any other suitable portion) of the gauge.

Although the gauge 140 can be coupled to the manipulator 100 in any suitable manner, some embodiments include a coupling mechanism 148 configured to couple the gauge to the manipulator between the first arm 110 and the second arm 120. Although the coupling mechanism can include any mechanism configured to effectuate such a coupling, some embodiments of the coupling mechanism are configured to allow the gauge to slide, twist, translate, or otherwise move along a proximal-distal axis. By way of non-limiting illustration, FIGS. 9A-9C show some embodiments of a manipulator 100 with a gauge 140 configured to slide distally (e.g., toward a first tip 112 and a second tip 122 of the manipulator 100) and proximally (e.g., toward a first handle 114 and a second handle 124 of the manipulator 100).

Where the gauge 140 is configured to slide distally or proximally, it can be so configured in any suitable manner. For example, some embodiments include a coupling mechanism 148 that allows the gauge to slide through the pivot 130 of the manipulator 100. Indeed, some embodiments of the coupling mechanism include one or more slots 147 configured to receive the pivot coupler 132 of the manipulator therethrough. Thus, some embodiments of the pivot coupler 132 not only couple the first arm 110 to the second arm 120 via the pivot 130, but further couple the gauge 140 to the manipulator (e.g., through the slot). By way of non-limiting illustration, FIG. 8 shows some embodiments of a gauge 140 with a coupling mechanism 148 that includes a slot 147 through which a pivot coupler (e.g., the pivot coupler 132 of FIG. 9A) is configured to extend. FIG. 9A shows an example of such a coupling.

According to some embodiments, the gauge 140 is biased in one or more directions (e.g., distally, proximally, or otherwise). For example, in some cases, the gauge is biased proximally such that while the instrument is not in use, the gauge extends proximally past at least one of the first handle 114 and the second handle 124 of the manipulator 100, thereby allowing a physician or other user to press the gauge distally (e.g., using a thumb, gravity, a spring, an elastic, a bias, or otherwise) to capture a measurement. For example, FIGS. 9A-9C show a manipulator 100 with a gauge 140, wherein the gauge is configured to be pressed distally (inward) once a first handle 114 and a second handle 124 have been pressed together to apply a spinal manipulation force (or at any other suitable time).

Where the gauge 140 is biased in one or more directions, the biasing can be created by any suitable mechanism. For example, some embodiments of the gauge include one or more biasing mechanisms 149, which can include any suitable compression springs, tension sprints, extension springs, leaf springs, disc springs, springs, coils, pressure chambers, memory foam, nitinol wires, rubber bands, elastic elements, or any other suitable component or components configured to bias one or more components toward or away from each other. By way of non-limiting illustration, FIG. 8 shows a gauge 140 having a biasing mechanism 149 in the form of a spring (not shown) configured to push against a pivot coupler (such as the pivot coupler 132 of FIG. 9A) to bias the gauge 140 proximally.

While the instrument can be configured to provide force to its tips 112, 122 in any suitable manner, in accordance with some embodiments, the first arm 110 and the second arm 120 are configured to produce an essentially constant force to the tips for a certain range of displacement of the tips. Indeed, in some cases, the desired or required amount of accuracy in applied force can help determine the lengths of the first arm 110 and the second arm 120 and the range of relative displacement of first tip and the second tip. For example, if the manipulation force needed or desired is for 100 newtons +/- 10 newtons, then the relationships of the arms and the tips can, in some cases, be adjusted for that amount of force accuracy.

As mentioned above, the gauge 140 (or gauges, as the case may be) can be configured to measure the distance between vertebrae (or any other objects) or any other suitable characteristics (e.g., displacement, force, or other characteristics) in any suitable manner. In some embodiments, the gauge is configured to convey a measurement of the distance to a user based on an amount the gauge moves distally when actuated distally by the user. For example, some embodiments include one or more indicia or scales 144, with different markings of such indicia indicating different distances (and thus, different implant sizes to be used). The indicia can be placed in any suitable location, such as on a body of the gauge, on a separate scale component, on an arm of the manipulator, on an external component (e.g., a ruler), on a proximal end of the gauge (or a distal end, middle, or other portion of the gauge), or anywhere else useful for assessing the output of the gauge. By way of non-limiting illustration, FIG. 9B shows some embodiments of a manipulator 100 with a gauge 140 having a scale 144 with three different markings, wherein the gauge 140 has a coupling mechanism 148 configured to allow the gauge 140 to slide distally with respect to the manipulator 100. In this example, the manipulator is configured to apply a specific amount of force when a practitioner presses the first arm 110 toward the second arm 120 (in some cases, all the way, such that the two arms (or one or more other contacts) touch). Then, the practitioner can press the gauge 140 inward (e.g., distally, such as by using a thumb), and depending on the distance between the first tip 112 and the second tip 122 (as determined by the distance between the first vertebra and the second vertebra, the gauge will move a certain distance distally, thereby aligning one of the markings of the indicia with the first handle 114 and second handle 124 (or any other suitable portion of the manipulators 100). Depending on the marking so aligned, the practitioner will be able to determine a distance (or displacement) resultant from the force applied, and thus be able to select an implant of appropriate size, stiffness, or other desired characteristics.

The gauge 140 can be configured to convey a measurement to a user in any suitable manner. In some embodiments, the result conveyed to the user is graded or continuous (e.g., showing results that fall between implant sizes), and in some embodiments the measurement conveyed is stepped, sloped, or quantized (e.g., showing a result corresponding with one or more particularly sized implants, such as the largest available implant that would not exceed the force applied by the manipulator 100). The measurement can be achieved in any suitable manner, but in some embodiments it is accomplished using a shape of an interface 150 between the gauge and the manipulator, as described in more detail below.

Some embodiments of the gauge 140 include one or more interfaces 150 configured to limit an amount the gauge is allowed to move based on the distance between or displacement of the vertebrae. The interface can be included on any suitable portion of the gauge, but in some cases, it is included on a distal end thereof. The interface can include any suitable component configured to correspond with the distance or displacement, but in some cases, it includes one or more shaped surfaces 151. Where a shaped surface is included, the shaped surface can have any suitable feature, size, shape, or other characteristic that allows them to limit the movement of the gauge (e.g., based on the distance between the first tip 112 and the second tip 122 of the manipulator 100). For example, some embodiments of the shaped surface 151 include one or more steps or stepped portions 151a, 151b, 151c, 151d, and some embodiments include one or more sloped, angled, or graded portions. In some cases, the shaped surface allows the gauge to move farther distally as the first tip and the second tip of the manipulator get farther apart. Thus, as a non-limiting example, where the first tip and the second tip move 1 mm apart as a result of the force applied by the manipulator, the gauge may be allowed to move a distance distally that causes the indicator 142 to indicate a value corresponding to 1 mm.

By way of non-limiting illustration, FIG. 8 shows a gauge 140 having an interface 150 on a distal end thereof, the interface including a shaped surface 151 (a stepped surface) such that the distal end is divided into multiple portions having increasingly larger diameters, including a first portion 151a having a first diameter, a second portion 151b having a second diameter that is larger than the first diameter, a third portion 151c having a third diameter that is larger than the second diameter, and a fourth portion 151d having a fourth diameter that is larger than the third diameter. As this relates to FIGS. 9A-9C, as the first tip 112 and the second tip 122 get farther away from each other, more of the interface 150 (shown in FIG. 8) is enabled to move past a distal end 152a of the recess 152 (FIG. 9A) formed in the first arm 110 and the second arm 120, with each portion (151a, 151b, 151c, 151d of FIG. 8) corresponding to an implant size, thereby changing which of the indicia of the scale (144 of FIG. 9B) aligns with the first handle 114 and the second handle 124, thereby indicating the implant size to a practitioner.

Importantly, the shaped surface 151 of the gauge 140 can include any suitable number of portions, having any suitable diameters, widths, or other features. Additionally, in some cases, a graded surface (instead of or in addition to a stepped surface) is used, thereby providing a continuous measurement as opposed to (or in addition to) a quantized one. Relatedly, the gauge can be configured to convey any suitable measurements (e.g., implant sizes) to the practitioner. In some embodiments, the gauge is provided to convey measurements between vertebral bodies (or other objects) in any suitable range, including between 0.1 mm and 50 mm, (e.g., between 5 mm and 10 mm for use in the cervical spine, between 7 mm and 18 mm for use in the lumbar spine, between 20 mm and 40 mm for use vertebrectomy/ corpectomy cases, or any other suitable measurements for any suitable use). For example, some embodiments of the gauge are configured to indicate that the disc height is 7 mm when a 100 newtons force is applied. In some embodiments, the gauge is configured to indicate that the disc height is 5 mm when a 50 newtons force is applied. In some cases, the gauge is configured to indicate disc heights between 4 mm and 20 mm and is related to applied forces of between 50 newtons and 150 newtons. Also, some embodiments are configured such that the gauge indicates disc heights of between 4 mm and 20 mm for an applied force per contact area of between 8 newtons and 20 newtons per square millimeter.

In some embodiments, the distal end of the gauge 140 is configured to be disposed between the first vertebra and the second vertebra or the tips 112, 122 while the measurement is taken. In some embodiments, the gauge is configured to measure the distance between the first vertebra and the second vertebra without contacting either the first vertebra or the second vertebra.

It is worth mentioning that some embodiments include multiple gauges 140, which may include gauges of different types or gauges that are configured to measure different characteristics. For example, some embodiments include a first gauge configured to measure force, and some embodiments include a second gauge configured to measure distance between vertebrae as a result of such force.

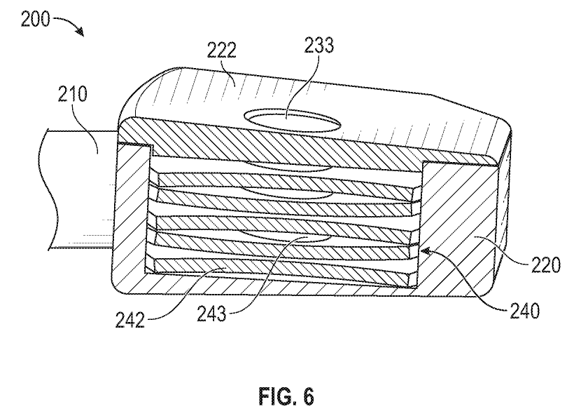

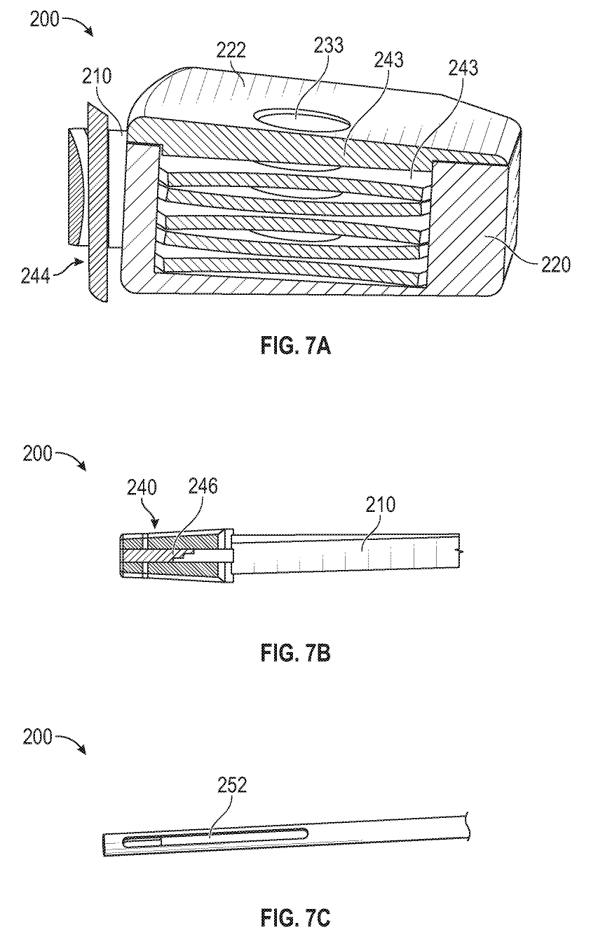

Turning now to FIGS. 6-7C, some additional embodiments of manipulation instruments are shown. In these illustrations, the instrument includes a manipulator 200 that is substantially in the form of a manipulation trial (instead of or in addition to a manipulator that is substantially in the form of calipers).

In some embodiments, the manipulator 200 optionally includes one or more housings 220. While the housing can include any component configured to form a particular shape or to house one or more other components, some embodiments of the housing are sized, shaped, and otherwise configured to be inserted into a spine. In some cases, the housing is configured to be inserted into a space between two vertebrae of the spine of the patient. Indeed, some iterations of the housing are configured to generally resemble a spinal implant trial. Some implementations include a trial arm 210, which, in some cases, is coupled to the housing. Although the housing can be formed of any material (including a rigid material), some embodiments of the housing include a pliable or flexible material, thereby allowing for the housing to be freely compressed. Thus, in some iterations, manipulation discs 242 (or leaves, squares, ovals, trapezoids, or any other suitable discs) (as discussed in greater detail below) provide substantially greater resistance to manipulation than the housing. In some embodiments, the housing comprises one or more rigid, semi-rigid, flexible, or other portions that are movable with respect to another portion (e.g., a lid (e.g., 222) that are movable with respect to another portion of the housing 220, for instance, as the discs 242 are compressed).

Some embodiments include a gauge 240. As with other embodiments described herein, the gauge can include any component suitable for measuring a manipulation force or conveying the measurement to a user. In particular, some embodiments of the gauge comprise one or more deformable, flexible, resilient, or movable elements (e.g., one or more springs (such as coil springs, compression springs, tension springs, leaf springs, Belleville springs, wave springs, shape-memory alloy springs, super-elastic springs, microfabricated springs, flat coil springs, spiral springs, constant force springs, elastomeric springs, magnetic springs, gas springs, fluid springs, or any other suitable springs), resilient members, elastics, deformable members, manipulation discs, or any other suitable component that can be compressed to allow the gauge to measure a manipulation force). Indeed, in some embodiments, the gauge includes one or more manipulation discs 242, which in some cases are disposed within the housing 220. Although some implementations have only a single manipulation disc, some embodiments include multiple manipulation discs (e.g., 2, 3, 4, 5, 6, 7, 8, 9, 10, or any other number). In some cases, the manipulation discs form one or more stack of discs. In some embodiments, the manipulation discs are resilient.

In some embodiments, one or more manipulation discs 242 include one or more curved, convex, concave, flat, cupped, undulated, waved, angled surfaces, or other surfaces that allow the discs to change shape or configuration when compressed. Indeed, some cases, one or more of the discs are configured to flex, flatten, or become flatter when compressed. To illustrate, in some implementations one or more of the manipulation discs generally resemble a plate (or a disc, leaf, star, rectangle, square, triangle, star, oval, ellipse, trapezoid, shape of an implant or a portion of an implant, polygon, scalloped polygon, Reuleaux polygon, circularly indented polygon, lobed shape, scalloped shape, wedge, kidney shape, asymmetrical shape, symmetrical shape, regular shape, irregular shape, ring, toroidal shape, sheet, substantially planar component, bowl, cup, or any other suitable component) that is bent, bowed, flattened, cupped, comprises a convex surface, comprises a concave surface, is flexed, is curved, is rippled, is undulated, or that otherwise forms a shape that is configured to change configuration when compressed. Thus, when the manipulation discs are compressed, the change in height of one or more of the manipulation discs (including a change in any particular manipulation disc on its own, or a change in a stack or collection of manipulation discs in the conglomerate) can be measured to determine a force. In some cases, the manipulation discs are pre-calibrated (e.g., the stiffness of the discs is known) to determine a precise force that correlates with a particular amount of change.

In some embodiments having multiple manipulation discs 242, a first manipulation disc and a second manipulation disc are situated (e.g., within the housing) so that the concave surface of the first manipulation disc faces a first direction and the concave surface of the second manipulation disc faces a second direction. For example, in some embodiments, the first disc faces with the concave curved surface pointed in a downward or posterior direction (e.g., away from a lid 222 of the housing 220) while the second disc faces with the concave curved surface pointed in an upward or anterior direction (e.g., toward the lid of the housing). In this manner, one or more gaps between the two discs (in a resting configuration) can be maximized so that a change in height of the stack, when compressed, is more pronounced. In some embodiments, multiple manipulation discs are directly adjacent to each other, or even in contact with one another, whereas in some instances, multiple manipulation discs are present within the housing but are separated from each other by a barrier or another component.

In some embodiments, the housing 220 includes one or more lids 222 or other openings. Indeed, in some cases, the housing includes a lid that is selectively removable. In some cases, the manipulation discs 242 are interchangeable, allowing for discs with different stiffnesses, curvatures, thicknesses, or other characteristics to be placed within the housing. In some instances, this configuration allows for different numbers of discs to be placed within the housing. Accordingly, in some cases, a single manipulator 200 is useful for measuring multiple different ranges of manipulation forces.

As stated above, some embodiments of the manipulation instrument include one or more deformable, flexible, resilient, or movable elements. In some cases, the deformable, flexible, resilient, or movable element includes a manipulation disc 242, configured to deflect when a spinal manipulation force is applied to the spine of the patient (e.g., in some cases a manipulation disc is configured to flatten or otherwise deform, deflect, or change to another configuration). In some embodiments, the gauge 240 is configured to measure the applied spinal manipulation force based on the deflection of the deformable, flexible, resilient, or movable element.

In some embodiments, the instrument includes indicia 244. For example, in some cases, the gauge includes an indicator. In some cases, a height of the housing serves as the indicator, or the housing itself functions as the indicator. In some cases, one or more characteristics of the manipulator 200 compared with indicia 244 serves as the indicator of the gauge 240. Accordingly, in some cases, the gauge 240 is configured to measure a change in height or other dimension of the stack of manipulation discs 242.

In some embodiments, the gauge 240 includes one or more pressure gauges 250 (e.g., a device having a pressure indicator 252 for more accurately measuring a manipulation force). While such a pressure indicator can function in any suitable manner, in some embodiments, the manipulator comprises an air tight chamber that is in fluid communication with the pressure sensor, such that when air pressure within the air tight chamber varies, so does the reading on the pressure indicator. Moreover, some embodiments include one or more integrated scales 246, hydraulic or pneumatic lifts configured to apply a known pressure, or other components for measuring a spinal manipulation force applied to a patient’s spine.

In some embodiments, when a desired spinal manipulation force is applied using the manipulator 200 (as determined by the gauge 240), a spinal implant of the proper size (e.g., the size that would apply the desired amount of manipulation force) is inserted into the space between two vertebrae of spine where the manipulation force was measured.

Referring now to all of FIGS. 1-7C, some embodiments of the systems and methods disclosed herein include a method for applying and measuring a spinal manipulation force. In some cases, the method includes one or more of the following: obtaining one or more manipulation instruments for applying one or more spinal manipulation forces (e.g., instruments including one or more manipulators 100, 200); obtaining one or more gauges 140, 240 for measuring the spinal manipulation force; applying the spinal manipulation force with the instrument; and measuring the spinal manipulation force with the gauge.

In some embodiments, the application of the spinal manipulation force with the instrument includes gradually increasing an application of the spinal manipulation force until the spinal manipulation force reaches a desired value, as measured with the gauge 140, 240. In some embodiments, the desired value is anywhere between 1 newton and 1,000 newtons (or any subrange thereof). For example, in some implementations, the desired value is approximately between 50 newtons and 250 newtons, and, in some implementations, the desired value is between approximately 75 newtons and approximately 150 newtons. Indeed, in some implementations, the desired value is between 8 newtons and 20 newtons per square millimeter of contact area.

In some embodiments, the method includes measuring a distance between two spinal vertebrae when the spinal manipulation force reaches the desired value. In some cases, the measuring a distance is done with a tool separate from the one used to apply the manipulation, but in some cases the same tool is used for applying the manipulation and measuring the distance (and, in some cases, measuring the manipulation force as well). In some cases, the method includes inserting an implant having a thickness approximately equal to the measured distance.

In some embodiments, the applying the spinal manipulation force includes inducing a deflection in a flexible material. In some embodiments, the measuring the manipulation force includes measuring the deflection in the flexible or resilient material and calculating the force based on one or more other measured characteristics (e.g., the stiffness and other characteristics) of the flexible material. In some embodiments, the method includes calibrating the instrument through application of a known force to determine the characteristics of the flexible material. In some embodiments, the method includes adjusting the stiffness of the flexible material. In some cases, the adjusting the stiffness includes varying a length of the flexible material, a cross-sectional area of the flexible material, or the material itself.

The systems and methods described herein may be modified in any suitable manner. Some embodiments of the disclosed systems and methods include a system for applying a desired amount of manipulation force to a patient’s spine. In some instances, the system includes an instrument configured to apply a trial force to a pair of vertebrae of the patient’s spine. While the instrument may be any suitable instrument (as discussed herein), in some embodiments, the instrument includes one or more of a flexible or resilient element (e.g., an arm 110, 120, a manipulation disc 242, or another flexible or resilient component) configured to deflect upon application of the trial force. In some embodiments, the instrument includes a gauge 140, 240 for measuring the trial force based on a degree of deflection of the flexible or resilient element. In some implementations, the system includes a spinal implant 500 configured to apply the desired amount of manipulation force to the patient’s spine, in accordance with the trial force as measured. In some cases, the system includes a plurality of spinal implants having differing characteristics (e.g., different thicknesses, different materials, different stiffnesses, etc.), thereby allowing a physician or practitioner to select a suitable spinal implant having the desired characteristics based on an applied manipulation force determined to be suitable through measurement of the force.

Some embodiments include one or more tools for measuring displacement. As an example, some embodiments include a tool for measuring the displacement of one or more spinal vertebrae. In some embodiments, the tool for measuring displacement shows a measurement of the displacement of spinal vertebrae at the point when the desired amount of spinal manipulation force is applied (e.g., when a gauge 140, 240 indicates that a desired value of force is being applied). Although the tool for measuring displacement can include any component suitable for measuring displacement, some embodiments include one or more gauges, indicators, indicia, alternative indicia, markings, rulers, scales, graduations, or any other components that may be useful for measuring displacement (such as any of the components discussed herein).

In some embodiments, the tool for measuring displacement is separate from a manipulation instrument (e.g., provided separately from a manipulator 100, 200, or a gauge 140, 240), but in some embodiments, the tool for measuring displacement is integrated with the manipulation instrument. As an example, FIGS. 4A-4B show a tool that includes alternative indicia 145 for measuring displacement. In FIG. 4A, the tips 112, 122 of the manipulator 100 are in a closed position, so the alternative indicia 145 do not show any displacement. In FIG. 4B, however, the tips are in an open position, and the tool for measuring displacement shows a corresponding shift in the indicia. Accordingly, in some embodiments once a desired manipulation force is obtained, the displacement can be measured, and an implant having a size that approximately corresponds to the measured displacement can be selected.

The various embodiments disclosed herein may be used separately or in combination with each other. As an example, in some embodiments a manipulation trial (e.g., the manipulator 200 of FIG. 6) is used in connection with a set of manipulation calipers (e.g., the manipulator 100 of FIG. 1). For instance, one or more tips (e.g., the first and second tips 112, 122 of FIG. 1) of a manipulator may include a manipulation gauge (e.g., the gauge 240 of FIG. 6) including one or more manipulation discs (e.g., 242 of FIG. 6), instead of or in addition to a deflection gauge (e.g., the gauge 140 of FIG. 1).

Additionally, it should be noted that in some cases, the deformable, flexible, movable, or resilient member or element is flexible but not resilient, such that when the member or element is deformed, it maintains its shape to allow for a manipulation force to be easily measured, even after the manipulation instrument has been removed from the patient. In some cases, however, the deformable, flexible, movable, or resilient member or element is both flexible and resilient such that the manipulation instrument returns to its original shape between uses and can be reused one or more times (e.g., many times following autoclaving).

As the systems and methods disclosed herein are compatible with one another, the systems discussed herein can be used in practicing the methods disclosed herein, and vice versa. Accordingly, the method may further include implementing, exercising, or otherwise using any of the components discussed herein for any of their stated or intended purposes, as reasonably predictable and understood by a person of ordinary skill in the art (e.g., using the described systems and methods with other objects besides vertebrae). The systems disclosed herein can be made in any suitable manner, and they may be used in any way consistent with their operational capabilities. Moreover, in some cases, any particular element or elements of any apparatus—or portion or portions of any method—disclosed herein can be omitted.

As used herein, the singular forms “a”, “an”, “the” and other singular references include plural referents, and plural references include the singular, unless the context clearly dictates otherwise. For example, reference to an indicator includes reference to one or more indicators, and reference to manipulation discs includes reference to one or more manipulation discs. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, cases, iterations, implementations, examples, figures, and illustrations set forth herein can be combined in any suitable manner with any other components or characteristics from one or more other embodiments, cases, iterations, implementations, examples, figures, and illustrations described herein. Indeed, all elements, embodiments, implementations, cases, examples, methods, steps, portions of methods, figures, and other portions of this disclosure can be: mixed and matched with each other in any suitable manner, replaced, substituted, omitted, rearranged, modified, performed in series, performed in parallel, performed in an overlapping manner, performed in a non-overlapping manner, or otherwise be modified to allow the described systems and methods to provide or measure a manipulation force.

What is claimed is:

1. An instrument for applying a spinal manipulation force and measuring a distance between vertebrae resulting from the spinal manipulation force, the instrument comprising:
a manipulator configured to apply the spinal manipulation force to a spine of a patient, the manipulator comprising:
a first arm having a first tip configured to interact with a first vertebra;
a second arm having a second tip configured to interact with a second vertebra; and
a pivot coupling the first arm to the second arm such that when the first arm is moved toward the second arm, the first tip is moved away from the second tip; and
a gauge for measuring a distance between the first vertebra and the second vertebra.

2. The instrument of claim 1, wherein the gauge is disposed between the first arm and the second arm.

3. The instrument of claim 1, wherein the gauge comprises a coupling mechanism that couples the gauge to the pivot.

4. The instrument of claim 1, wherein the gauge is configured to move along a distal-proximal axis.

5. The instrument of claim 4, wherein the gauge is biased proximally.

6. The instrument of claim 4, wherein the gauge is configured to move a first distance distally when the first tip has a first displacement from the second tip and a second distance distally when the first tip has a second displacement from the second tip.

7. The instrument of claim 1, wherein the gauge is at least partially disposed within a recess formed by at least one of the first arm and the second arm.

8. The instrument of claim 1, wherein the first arm is configured to contact the second arm when the first arm and the second arm are moved toward each other.

9. The instrument of claim 1, wherein a distal end of the gauge comprises a stepped surface.

10. The instrument of claim 1, wherein a proximal end of the gauge comprises indicia configured to convey the distance between the first vertebra and the second vertebra as measured to a user.

11. The instrument of claim 1, wherein the first tip is configured to abut an endplate of the first vertebra, and wherein the second tip is configured to abut an endplate of the second vertebra.

12. A method for applying a spinal manipulation force and measuring a distance between vertebrae resulting from the spinal manipulation force, the method comprising:
obtaining an instrument comprising:
a manipulator configured to apply the spinal manipulation force to a spine of a patient, the manipulator comprising:
a first arm having a first tip configured to interact with a first vertebra;
a second arm having a second tip configured to interact with a second vertebra; and
a pivot coupling the first arm to the second arm such that when the first arm is moved toward the second arm, the first tip is moved away from the second tip; and
a gauge;
applying the spinal manipulation force; and
measuring a distance between the first vertebra and the second vertebra using the gauge.

13. The method of claim 12, further comprising selecting an interbody implant based on the distance between the first vertebra and the second vertebra as measured.

14. The method of claim 12, further comprising installing the interbody implant between the first vertebra and the second vertebra.

15. The method of claim 12, wherein the applying the spinal manipulation force and the measuring the distance between the first vertebra and the second vertebra using the gauge are performed simultaneously.

16. The method of claim 12, wherein the measuring the distance between the first vertebra and the second vertebra using the gauge comprises moving the gauge distally between the first tip and the second tip.

17. The method of claim 12, wherein the manipulator is configured to apply the spinal manipulation force in a known, set value when the first arm is placed into contact with the second arm.

18. An instrument for applying a spinal manipulation force and measuring a distance between vertebrae resulting from the spinal manipulation force, the instrument comprising:
a manipulator configured to apply the spinal manipulation force to a spine of a patient; and
a gauge configured to measure a distance between a first vertebra and a second vertebra during application of the spinal manipulation force.

19. The instrument of claim 18, wherein the gauge is configured to move along a distal-proximal axis, and wherein the gauge comprises an interface configured to affect an amount the gauge is allowed to move distally depending on the distance between the first vertebra and the second vertebra during application of the spinal manipulation force.

20. The instrument of claim 18, wherein the gauge is configured to measure the distance between the first vertebra and the second vertebra without contacting either the first vertebra or the second vertebra.
